(19) United States
(12) Patent Application Publication
(10) Pub. No.: US 2005/0101023 A1
(43) Pub. Date: May 12, 2005

(54) Methods for diagnosing urinary tract and prostatic disorders

(76) Inventors: James A. Rogers, Ivoryton, CT (US); Alexander F. Rosenberg, Brookline, MA (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/812,753

(22) Filed: Mar. 29, 2004

Related U.S. Application Data
(60) Provisional application No. 60/458,850, filed on Mar. 28, 2003.

(51) Int. Cl. G01N 33/92

(52) U.S. Cl. 436/71

(57) ABSTRACT

Disclosed are methods of detecting urinary tract and prostatic disorders using reference profiles. Also disclosed are methods of identifying agents for treating urinary tract and prostatic disorders.

### RELATED APPLICATIONS

This application claims priority to U.S. Ser. No. 60/458,850 filed Mar. 28, 2003 which is incorporated herein by reference in its entirety.

### FIELD OF THE INVENTION

The invention relates to diagnostic techniques and more particularly methods for diagnosing urinary tract and prostatic disorders.

### BACKGROUND OF THE INVENTION

Urinary tract disorders (UTDs) are a category of disorders that have diverse mechanisms of onset and progression. UTD include interstitial cystitis (IC), a chronic inflammatory condition of the bladder wall that frequently goes undiagnosed. The cause of IC is not well understood and no treatment is uniformly effective for all patients. There are two types of IC: non-ulcerative IC, which affects 90 percent of IC sufferers, and ulcerative IC (presenting with Hunner's patches or ulcers). IC is frequently misdiagnosed as an acute urinary tract infection (cystitis), a disorder that can be successfully treated with antibiotics.

### SUMMARY OF THE INVENTION

The invention is based on the discovery of a profile of analytes that are correlated with urinary tract disorders. Urinary tract disorders include, for example, interstitial cystitis, prostatitis, kidney infection or inflammation, urethritis, prostrate hypertrophy, and urinary tract stones. The analytes that are differentially present in urinary tract disorders are referred to herein as “UTD-X,” or UTD-X analytes. These analytes, as well as metabolites, derivatives, homologs, analogs and fragments thereof, will hereinafter be collectively designated as UTD-X, where X is an integer between 1 and 95. A profile containing the relative levels of one or more UTD-X members is known as a urinary tract disorder reference profile.

The invention provides a urinary tract disorder reference profile that includes a pattern of one or more analytes or metabolites thereof of UTD 1-95. For example, a urinary tract disorder reference profile includes a pattern of one or more analytes or metabolites of UTD 3, 6, 8-11 and 18. The urinary tract disorder reference profile additionally includes a pattern of one or more analytes or metabolites of UTD 24-95. Alternatively, the urinary tract disorder reference profile includes a pattern of one or more analytes or metabolites of UTD 6, and 25-55 or UTD 3, 8-11, 18, 56-95.

The invention also provides a method of metabolomically predicting whether a subject is predisposed to developing a urinary tract disorder by obtaining a urinary tract disorder reference profile from the subject and comparing the urinary tract disorder reference profile with a control urinary tract disorder reference profile.

The invention further provdes methods of identifying markers indicative of a urinary tract disorder in a subject by determining the levels of one or more analytes or metabolites thereof in a subject sample and determining those analytes or metabolites that are present in a different concentration in the subject sample compared to a control sample. The presence of the analytes or metabolites at a different concentration is indicative of a urinary tract disorder in the subject. The analytes are, for example, UTD 3, 6, 8-11, and 18.

The invention also provides methods of diagnosing a urinary tract disorder (UTD), or a predisposition to developing a urinary tract disorder in a subject by determining a level of a UTD-associated analyte in a subject derived sample. An alteration, e.g., an increase or a decrease of the level compared to a normal control level indicates that the subject suffers from or is at risk of developing a urinary tract disorder. A UTD-associated analyte is an analyte that is characterized by being present at a level that differs in a biological sample obtained from an individual with a urinary tract disorder compared to a biological sample obtained from a normal (control) individual. A normal control individual is a healthy individual or population of individuals known not to be suffering from a urinary tract disorder. For example, a control level is a database of patterns from previously tested individuals. A normal individual is one with no clinical symptoms of a urinary tract disorder. A UTD-associated analyte is one or more of UTD 1-95.

The level of the analyte is increased 1.1-fold, 1.25-fold, 1.5-fold, 1.75-fold, 2-fold, 5-fold, 10-fold, 25-fold, 100-fold or more over than the normal control level. Alternatively, the level of analyte is decreased 10%, 15%, 25%, 50%, 75%, 90%, 95%, 99%, 99.9% or 99.99% or more compared to the control level.

The subject derived sample is any sample from a test subject, e.g., a patient known to or suspected to have a urinary tract disorder. For example, the sample is urine, prostatic fluid, or urinary tract tissue.

The invention firther provides methods of assessing the efficacy of a treatment of a urinary tract disorder in a subject, by determining a level of a UTD-associated analyte in a subject derived sample, and comparing the level to a normal control level. The subject has been treated for a urinary tract disorder.

In another aspect the invention provides methods of identifying an agent that modulates the onset or progression of a urinary tract disorder in a subject. The method includes contacting the subject with a candidate agent, and determining a test level of an analyte in a sample derived from the subject. The test level is compared with a reference level of the analyte. An alteration, e.g., an increase or decrease of the test level relative to the reference level, indicates that the test agent modulates the onset or progression of a urinary tract disorder. The reference level is derived from a sample from the subject. Alternatively, the reference level is derived from a database.

Also included in the invention is a kit having a detection reagent that identifies one or more of UTD 1-95.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

One advantage of the methods described herein is that the disease is identified prior to detection of overt clinical symptoms of UTDs. Other features and advantages of the invention will be apparent from the following detailed description, and from the claims.

### BRIEF DESCRIPTION OF THE FIGURES

FIG. 1 is a one-dimensional scatterplot of the normalized analyte signal (for hydroxyproline), demonstrating the increased level of hydroxyproline in urine samples from interstitial cystitis patients as compared to unaffected individuals.

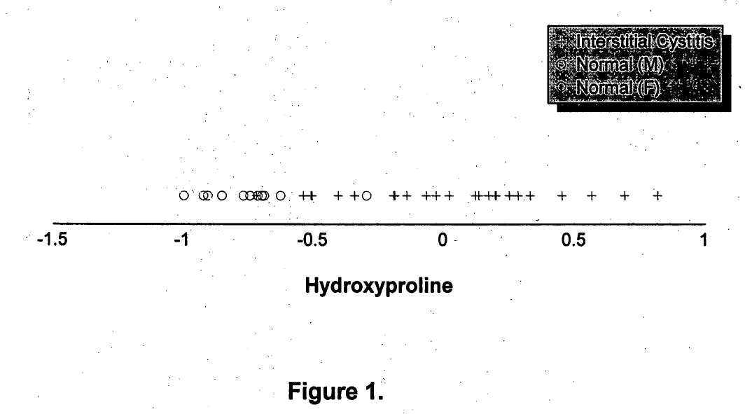

FIG. 2 is a two-dimensional scatterplot showing the normalized analyte signals for glutamic acid and hydroxyproline, demonstrating that the levels of hydroxyproline and glutamic acid are increased in urine samples from interstitial cystitis patients as compared to unaffected individuals, and that these two analytes combined provide better separation than either alone.

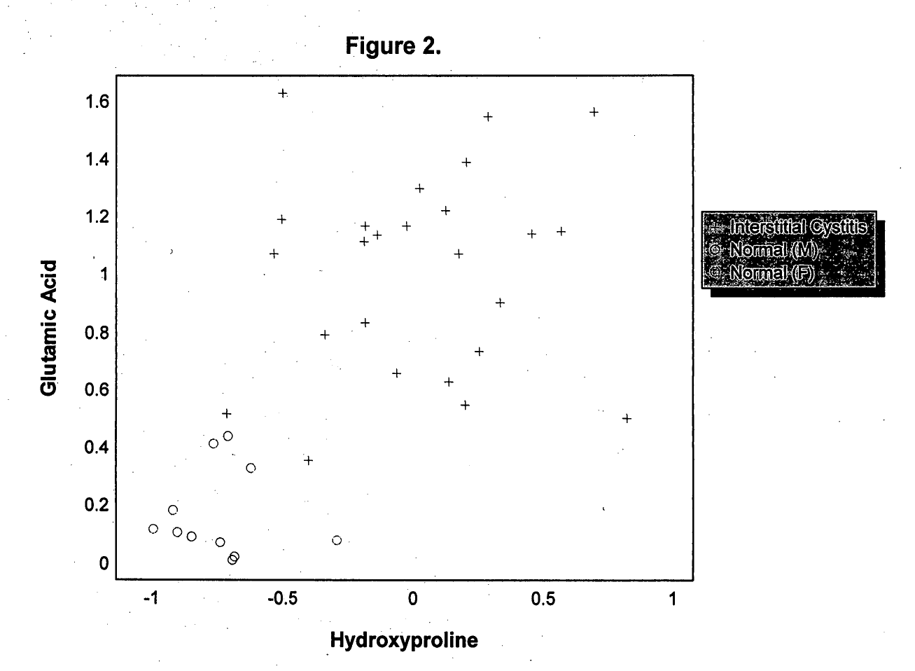

FIG. 3 includes twenty-three one-dimensional scatterplots of normalized analyte signals that make up an analyte profile of the invention.

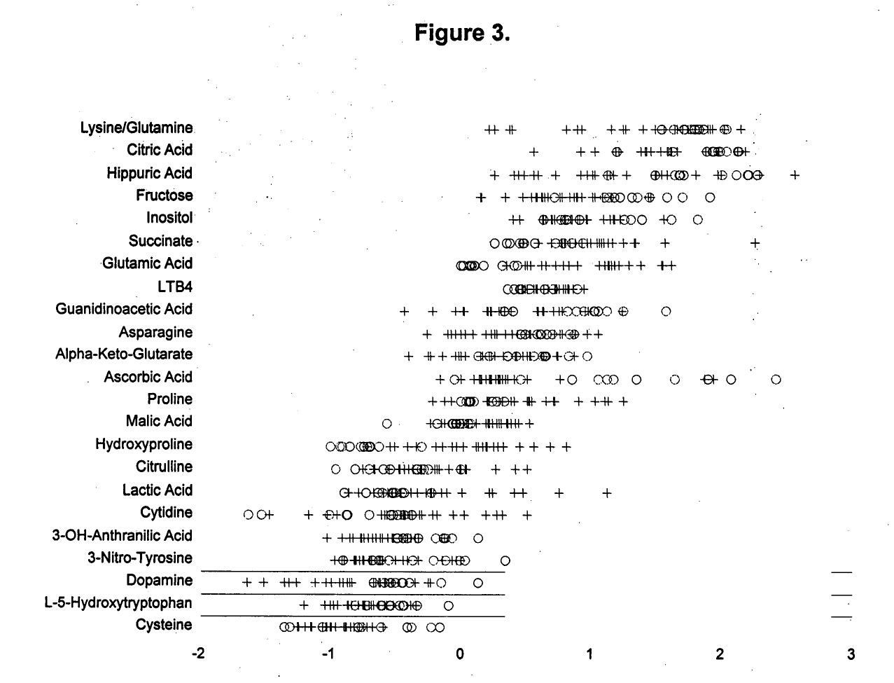

FIG. 4 is a one-dimensional scatterplot of the derived diagnostic index. This index is a weighted average of normalized signals that make up an analyte profile of the invention. The separation of interstitial cystitis samples and normal samples demonstrates the diagnostic utility of the invention.

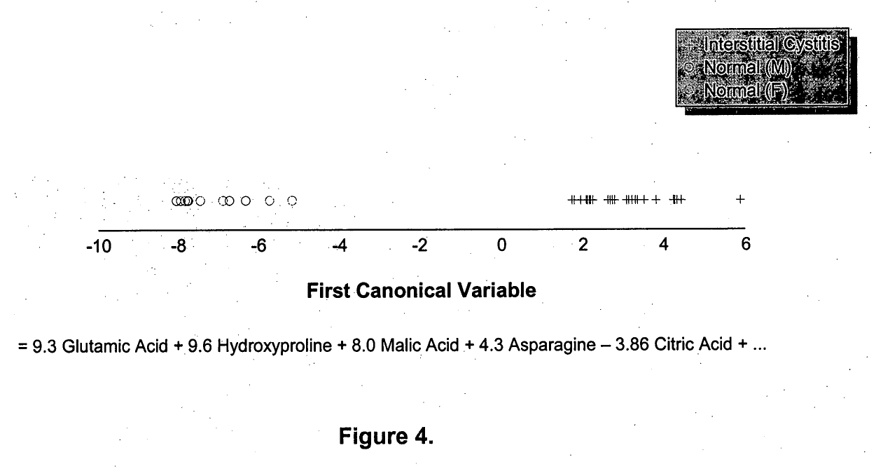

### DETAILED DESCRIPTION

The present invention is based, in part, on the discovery of changes of analyte levels in urine samples from interstitial cystitis (IC) patients. The differences in analyte levels were identified by analyzing the relative concentrations of large sets of small molecules using mass spectrometry to create biochemical profiles for individual samples. Such profiles are then compared to identify biochemical changes that occur in IC patients as compared to unafflicted patients. Statistical and bioinformatic analyses of these profiles identify patterns of change for the small molecules measured. These patterns of change form the basis for biochemical signatures that are characteristic for urinary tract disorders. These signatures are used to predict the presence and progression of urinary tract disorders, as well as the toxicological and clinical behavior of new drug candidates to treat or prevent urinary tract disorders.

The analyte profiles were generated from two separate experiments using samples from human subjects that were either suffering from interstitial cystitis or control subjects that were not suffering from interstitial cystitis. In the first experiment, 36 human subjects were analyzed (25 from patients with interstitial cystitis; 11 from control subjects). By comparing analyte patterns between patients diagnosed with IC and normal patients, 9 analytes were identified as being commonly increased in IC and 24 analytes were identified as being commonly decreased in IC. (See, Table 1A) The second experiment comprised a larger subject sample size and therefore is more statistically significant. By comparing analyte patterns between patients diagnosed with IC and normal patients, 33 analytes were identified as being commonly increased in IC and 46 analytes were identified as being commonly decreased in IC. (See, Table 1B). Seven analytes, glutamic acid, lysine/glutamine, CAP54, ascorbic acid, citric acid, malic acid and lactic acid, were shared between the data sets from the two experiments. The differentially modulated analytes identified herein are used for diagnostic purposes as markers of urinary tract disorders.

The analytes whose levels are modulated (i.e., increased or decreased) in IC patients are summarized in Table 1 and are collectively referred to herein as “UTD-associated analytes.” Unless indicated otherwise, “UTD” is meant to refer to any of the analytes disclosed herein (e.g., UTD 1-95). The analytes that have been previously described are identified by chemical name. For those analytes that have not heretofore been described are identified by parent, daughter mass and collision energy. Exemplary separation conditions are described in the Examples below. With this information those skilled in the art can readily identify a UTD-associated analyte in a sample. For example, parent and daughter masses, and collision energies are used to set up a mass spectrometer. The column type and the mobile phase conditions described in the Examples are used to set up the HPLC step. For the majority of UTD-associated analytes, a single peak that changes with IC will be visible in the chromatogram from human urine. Where more than one peak is visible, the desired peak is the one that shows a change between diseased and normal samples.

TABLE 1A
Relative analyte composition in IC-affected vs.
unaffected subjects (Experiment 1)

| | | Affected level | | | |
|---|---|---|---|---|---|
| | | relative to | | Fragment | Collision |
| UTD # | Chemical name | Unaffected level | Analyte Mass | Mass | Energy |
| 1 | Hydroxyproline | greater | 132 | 68 | 30 volts |
| 2 | Citrulline | greater | 174 | 131.2 | −22 volts |
| 3 | Lactic Acid | greater | 89.2 | 43.1 | −17 volts |
| 4 | Cytidine | greater | 243.9 | 111.8 | 15 volts |
| 5 | Succinate | greater | 117.1 | 73 | −15 volts |
| 6 | Glutamic Acid | greater | 147.9 | 102 | 17 volts |
| 7 | LTB4 | greater | 335.4 | 195.3 | −23 volts |
| 8 | Malic Acid | greater | 133 | 114.9 | −20 volts |
| 9 | CAP54 | greater | 115.9 | 69.9 | 18 volts |
| 10 | Lysine/Glutamine | less | 146.9 | 130 | 16 volts |
| 11 | Citric Acid | less | 191 | 87 | −25 volts |
| 12 | Hippuric Acid | less | 177.9 | 133.9 | −16 volts |
| 13 | Fructose | less | 179 | 89.2 | −15 volts |
| 14 | Inositol | less | 179 | 161 | −18 volts |
| 15 | Guanadinoacetic | less | 118 | 101 | 15 volts |
| | Acid | | | | |
| 16 | Asparagine | less | 132.9 | 74 | 22 volts |
| 17 | Alpha-Keto- | less | 144.8 | 101 | −15 volts |
| | Glutarate | | | | |
| 18 | Ascorbic Acid | less | 175.1 | 115.1 | −17 volts |
| 19 | 3-OH-Anthranilic | less | 154 | 136 | 15 volts |
| | Acid | | | | |
| 20 | 3-Nitro-Tyrosine | less | 226.9 | 180.8 | 29 volts |
| 21 | Dopamine | less | 153.9 | 136.7 | 17 volts |
| 22 | L-5- | less | 221 | 203.8 | 17 volts |
| | Hydroxytryptophan | | | | |
| 23 | Cysteine | less | 122 | 76 | 20 volts |

TABLE 1B
Relative analyte composition in IC-affected vs.
unaffected subjects (Experiment 2)

| | | Affected level | | | |
|---|---|---|---|---|---|
| | | relative to | Analyte | Fragment | Collision Energy |
| UTD # | Chemical name | Unaffected level | Mass | Mass | (in volts) |
| 24 | 1-methylhistamine | greater | 126 | 109 | 20 |
| 25 | Acetylcholine | greater | 146 | 86.7 | 20 |
| 26 | Allantoin | greater | 159 | 116 | 10 |
| 27 | CAP102 | greater | 227.3 | 114.1 | 13 |
| 28 | CAP108 | greater | 229.1 | 142.2 | 31 |
| 29 | CAP133 | greater | 212 | 124.1 | 26 |
| 30 | CAP139 | greater | 246.2 | 85.1 | 29 |
| 31 | CAP153 | greater | 245.9 | 114.1 | 22 |
| 32 | CAP175 | greater | 269.3 | 110.3 | 35 |
| 33 | Creatinine | greater | 113.9 | 44 | 26 |
| 6 | Glutamic Acid | greater | 147.9 | 102 | 17 |
| 34 | CAP188 | greater | 356.8 | 167 | −22 |
| 35 | CAP231 | greater | 295.9 | 164 | −25 |
| 36 | CAP261 | greater | 278.8 | 196.9 | −19 |
| 37 | 4-Pyridoxic Acid | greater | 182.1 | 138.1 | −21 |
| 38 | CAP277 | greater | 224.9 | 197 | −18 |
| 39 | CAP278 | greater | 135.1 | 75 | −20 |
| 40 | CAP279 | greater | 182 | 108.1 | −29 |
| 41 | CAP282 | greater | 118.9 | 75 | −18 |
| 42 | CAP283 | greater | 167 | 123.9 | −21 |
| 43 | CAP284 | greater | 173.1 | 80 | −34 |
| 44 | CAP287 | greater | 225 | 127 | −26 |
| 45 | CAP309 | greater | 210.9 | 151.2 | −13 |
| 46 | CAP317 | greater | 199.1 | 99 | −21 |
| 47 | CAP320 | greater | 254.9 | 145.1 | −25 |
| 48 | CAP337 | greater | 341 | 149 | −33 |
| 49 | CAP345 | greater | 215.8 | 136 | −23 |
| 50 | CAP349 | greater | 195 | 89 | −29 |
| 51 | CAP351 | greater | 193.1 | 92.2 | −26 |
| 52 | Hypoxanthine | greater | 135 | 92.1 | −23 |
| 53 | Kynurenic Acid | greater | 188 | 144 | −40 |
| 54 | Uracil | greater | 111 | 42.2 | −22 |
| 55 | Xanthine | greater | 151 | 108 | −24 |
| 56 | CAP126 | less | 263 | 99.1 | 17 |
| 57 | CAP12 | less | 326.9 | 81.1 | 33 |
| 58 | CAP15 | less | 350.9 | 187 | 20 |
| 59 | CAP16 | less | 466.9 | 244.9 | 23 |
| 60 | CAP17 | less | 491.1 | 245 | 23 |
| 61 | CAP18 | less | 509.1 | 263.1 | 30 |
| 62 | CAP19 | less | 515.1 | 269.1 | 25 |
| 63 | CAP2 | less | 204.3 | 84.9 | 25 |
| 64 | CAP4 | less | 244.9 | 80.9 | 23 |
| 65 | CAP7 | less | 261 | 96.7 | 14 |
| 66 | CAP9 | less | 268.9 | 105.1 | 20 |
| 67 | Choline | less | 104 | 60.1 | 15 |
| 68 | Glycine | less | 75.8 | 30.2 | 21 |
| 69 | Guanidinoacetic Acid | less | 118 | 101 | 15 |
| 70 | Isoleucine | less | 132 | 69 | 25 |
| 71 | Leucine | less | 132 | 44.1 | 34 |
| 10 | Lysine/Glutamine | less | 146.9 | 130 | 16 |
| 72 | Methionine | less | 149.9 | 103.9 | 17 |
| 9 | CAP54 | less | 115.9 | 69.9 | 18 |
| 73 | Serine | less | 105.9 | 60.1 | 15 |
| 74 | Valine | Less | 117.9 | 72 | 27 |
| 18 | Ascorbic Acid | less | 175.1 | 115.1 | −17 |
| 75 | CAP187 | Less | 189.1 | 129.1 | −16 |
| 76 | CAP198 | Less | 189 | 106.9 | −29 |
| 77 | CAP203 | Less | 172.7 | 84.7 | −17 |
| 78 | CAP205 | Less | 194 | 150.2 | −15 |
| 79 | CAP211 | Less | 288.9 | 96.9 | −47 |
| 80 | CAP215 | Less | 192.2 | 111.9 | −17 |
| 81 | CAP222 | Less | 272.1 | 186.8 | −15 |
| 82 | CAP223 | Less | 179 | 89.2 | −14 |
| 83 | CAP262 | Less | 199.8 | 163.2 | −29 |
| 84 | CAP27 | Less | 201.1 | 121.3 | −31 |
| 11 | Citric Acid | Less | 191 | 87 | −25 |
| 85 | Isocitric Acid | Less | 191 | 172.9 | −18 |
| 8 | Malic Acid | Less | 133 | 114.9 | −20 |
| 86 | Mevalonic Acid Lactone | Less | 147.2 | 59.3 | −19 |
| 87 | CAP263 | Less | 186.5 | 106.6 | −32 |
| 88 | CAP275 | Less | 186.9 | 81.1 | −21 |
| 89 | CAP27 | Less | 201 | 121.2 | −30 |
| 90 | CAP300 | Less | 399.3 | 187.1 | −10 |
| 91 | CAP301 | Less | 187.8 | 107.3 | −36 |
| 92 | CAP318 | Less | 360.7 | 187 | −10 |
| 93 | CAP324 | Less | 269.1 | 144.9 | −28 |
| 94 | CAP331 | Less | 200.2 | 157.9 | −21 |
| 95 | CAP353 | Less | 198 | 73.9 | −23 |
| 3 | Lactic Acid | Less | 89.2 | 43.1 | −12 |

The term “analyte” includes organic and inorganic molecules that are present in the tissue, fluid, cell, cellular compartment, or organelles. An analyte includes signaling molecules and intermediates in the chemical reactions that transform energy derived from food into usable forms. The term “metabolite” includes any chemical or biochemical product of a metabolic process, such as any compound produced by the processing, cleavage or consumption of a biological molecule (e.g., a protein, carbohydrate, or lipid). The term “metabolome” includes all of the analytes present in a given organism. The metabolome includes metabolites as well as products of catabolism.

By measuring the level of the various analytes in a sample, urinary tract disorders are diagnosed. Similarly, measuring the level of these analytes in response to various agents will identify agents for treating urinary tract disorders.

The invention involves determining (e.g., measuring) the level of at least one, and up to all the analytes listed in Table 1. Optionally, the UTD-associated analyte is determined in a sample by detecting one or more metabolites of the analyte in the sample. Using molecular mass information and collision energy provided herein, the UTD associated-analytes are detected and measured using techniques well known to one of ordinary skill in the art. For example, UTDs 1-95 are detected by mass spectrometric analysis.

The level of one or more of the UTD-associated analytes in the test population, e.g., a patient derived sample, is then compared to levels of the same analytes in a reference population. The reference population includes one or more samples for which the compared parameter is known, ie., urinary tract disorder sample or normal (non-urinary tract disorder sample).

Whether or not a pattern of analyte levels in the test population (e.g. patient derived sample) compared to the reference population (i.e, control sample) indicates UTD or predisposition thereto depends upon the composition of the reference population. For example, if the reference cell population is composed of a non-UTD sample (i.,e, derived from a subject or subjects known not to be suffering from a urinary tract disorder), a similar analyte pattern in the test population and reference population indicates the test population is non-UTD. Conversely, if the reference population is made up of a UTD sample, a similar analyte pattern between the test population and the reference population indicates that the test population includes UTD.

A level of expression of a UTD analyte in a test population is considered altered in levels if the level varies from the reference population by more than 0.5, 1.0, 1.5, 2.0, 5.0, 10.0 or more fold from the level of the corresponding UTD analyte in the reference population.

Differential analyte levels between a test population and a reference population are normalized to a control analyte or a control composite analyte value. For example, a control analyte includes an analyte that does not differ between affected and unaffected members of a tested population. For example, the control analyte is creatinine. The control composite analyte value is a composite of many analytes that together provide a measurement that is expected not to change with disease state. A control composite analyte, for example, is the mean or median of the distribution of each analyte tested in a population.

The test population is compared to multiple reference populations. Each of the multiple reference populations differs in the known parameter. Thus, a test population is compared to a second reference population known to contain, e.g., UTD, as well as a second reference population known to contain, e.g., non-UTD (normal).

The test sample is obtained from a bodily tissue (e.g., from kidney or bladder) or a bodily fluid, e.g., biological fluid (such as urine, or prostatic fluid). For example, the test sample is purified from urinary tract tissue. The sample is obtained from the entire tissue, entire cell or from specific cellular compartments such as the cytoplasm, the mitochondria, the Golgi apparatus, the endoplasmic reticulum, the nucleus, the chloroplasts, the cytosol. The sample is substantially free of macromolecules (e.g., large proteins and polynucleotides with molecular weights of greater than 10,000).

The reference population is derived from a tissue or fluid type similar to the test population. Optionally, the control population is derived from a database of molecular information from samples for which the assayed parameter or condition is known.

The subject is preferably a mammal. The mammal can be, e.g., a human, non-human primate, mouse, rat, dog, cat, horse, or cow.

Analytes disclosed herein are detected in a variety of ways known to one of skill in the art, including the refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), Light Scattering analysis (LS), Mass Spectrometry, Pyrolysis Mass Spectrometry, Nephelometry, Dispersive Raman Spectroscopy, gas chromatography combined with mass spectroscopy, liquid chromatography combined with mass spectroscopy, MALDI combined with mass spectroscopy, ion spray spectroscopy combined with mass spectroscopy, capillary electrophoresis, NMR and IR detection.

Diagnosing Urinary Tract Disorders

A urinary tract disorder is diagnosed or the risk of developing a urinary tract disorder is determined by measuring the level of one or more UTD-associated analytes from a test population (i.e., a patient derived sample such as urine, prostatic fluid or urinary tract tissue). Expression of one or more UTD-associated analytes, e.g., UTD 1-95 is determined in the test sample and compared to the expression of the normal control level. Preferably, expression of one or more UTD-associated analytes including UTD 3, 6, 8-11 or 18 are measured.

A normal control level is a profile of UTD-associated analytes typically found in a population known not to be suffering from UTD. An increase or a decrease of the level of expression in the patient derived tissue sample of the UTD-associated analytes indicates that the subject is suffering from or is at risk of developing UTD. For example, an increase of UTD 6 and 24-55 indicates that the subject is suffering from or is at risk of developing a UTD. In contrast, a decrease of UTD 3, 8-11, 18, 56-95 indicates that the subject is suffering from or is at risk of developing a UTD

An alteration of one or more of the UTD-associated analytes in the test population compared to the normal control level indicates that the subject suffers from or is at risk of developing UTD. For example, at least 1%, 5%, 25%, 50%, 60%, 80%, 90% or more of the panel of UTD-associated analytes (e.g., UTD1-95), are altered.

Assessing Efficacy of Treatment of UTD in a Subject

The UTD-associated analytes identified herein also allow for the course of treatment of UTD to be monitored. In this method, a test population is provided from a subject undergoing treatment for UTD. If desired, test cell populations are obtained from the subject at various time points before, during, or after treatment. Expression of one or more of the UTD-associated analytes in the population is then determined and compared to a reference population which includes samples whose UTD state is known. The reference population has not been exposed to the treatment.

If the reference population contains a non-UTD sample, a similarity in expression between UTD-associated analyte in the test population and the reference population indicates that the treatment is efficacious. However, a difference in expression between UTD-associated analyte in the test population and a normal control reference population indicates a less favorable clinical outcome or prognosis.

By “efficacious” is meant that the treatment leads to a reduction in a pathologically increased analyte or an increase of a pathologically decreased analyte or a decrease in size, or prevalence, of petechial hemorrhages or Hunner's patches in a subject. When treatment is applied prophylactically, “efficacious” means that the treatment retards or prevents a UTD from forming or retards, prevents, or alleviates a symptom of clinical UTD. Assessment of UTD is made using standard clinical protocols. Efficaciousness is determined in association with any known method for diagnosing or treating UTD. UTD is diagnosed for example, by identifying symptomatic anomalies, e.g., bladder pain, urinary urgency, suprapubic or perineal pain and pressure. Prostatitis is diagnosed, for example, by digital evaluation of the prostate; urinalysis may show an increased white blood cell count; and a blood test will measure levels of prostate-specific antigen (PSA).

Assessing the Prognosis of a Subject with UTD

Also provided is a method of assessing the prognosis of a subject with UTD by comparing the level of one or more UTD-associated analytes in a test population to the level of the analytes in a reference population derived from patients over a spectrum of disease stages. By comparing the analyte level of one or more UTD-associated analytes in the test population and the reference population(s), or by comparing the pattern of analyte levels over time in test populations derived from the subject, the prognosis of the subject can be assessed.

For example, when the control sample, (i.e., reference profile) is derived for a subject sample known to be suffering from a UTD a decrease in expression of one or more of UTD 6, 24-55 compared to a control or an increase of expression of one or more of UTD 3, 8-11, 18, 56-95 compared to a normal control indicates more favorable prognosis. Alternatively, when the control sample, (i.e., reference profile) is derived for a subject sample known not to be suffering from a UTD, e.g., a normal control sample) an increase in expression of one or more of UTD 6, 24-55 or a decrease in expression of one or more of UTD 3, 8-11, 18, 56-95 indicates a less favorable prognosis for the subject.

Kits

The invention also includes a UTD associated analyte-detection reagent in the form of a kit. For example, the kit includes a labeled compound or agent that detects the UTD-associated analyte in a biological sample. The kit further includes a means for determining the amount of the analyte in the sample (e.g., an antibody, molecular or chemical sensor against the UTD associated analyte). Optionally, the kit contains, e.g., a buffering agent, a preservative, a stabilizing agent, or components necessary for detecting the detectable agent (e.g., a substrate). The kit contains a control sample or a series of control samples that is assayed and compared to the test sample contained. Each component of the kit is enclosed within an individual container and all of the various containers are within a single package along with instructions for observing whether the tested subject is suffering from or is at risk of developing a disorder associated with the UTD-associated analyte. For example, the kit comprises two or more of UTD1-95 along with detection means and instructions for use thereof.

This invention is further illustrated by the following examples that should not be construed as limiting. The contents of all references and published patents and patent applications cited throughout the application are hereby incorporated by reference.

### EXAMPLE 1

### Detecting UTD-Associated Analytes

UTD-associated analytes or metabolites are detected using a single technique or a combination of techniques for separating and/or identifying analytes known in the art. Examples of separation and analytical techniques which are used to separate and identify the UTD-associated analyte in a sample include mass spectroscopy (MS), HPLC, TLC, electrochemical analysis, refractive index spectroscopy (RI), Ultra-Violet spectroscopy (UV), fluorescent analysis, radiochemical analysis, Near-InfraRed spectroscopy (Near-IR), Nuclear Magnetic Resonance spectroscopy (NMR), and Light Scattering analysis (LS). The methods of the invention detect both electrically neutral as well as electrochemically active compounds. Preferably, the separation and detection of UTD associated analytes is accomplished by MS. Detection and analytical techniques are arranged in parallel to optimize the number of molecules identified.

For example mass spectroscopy is used as a method for detecting and quantifying the analytes contained in a biological source (e.g., urine) taken from a subject. The analytes from a subject are separated through the use of column chromatography. Multiple columns are used as shown in Table 2, with each column designed to separate classes of compounds. In this format, there is a column-switching valve which allows for staggered injections into the multiple columns. This format is described below and in U.S. patent application Ser. No. 10/323,493, the contents of which are incorporated by reference herein.

TABLE 2
Column chromatographic separation of analytes

| Column # | Column type | Aqueous Mobile Phase | Organic Mobile Phase |
|---|---|---|---|
| 1 | Phenomenex Luna | 0.1% acetic acid in | 0.1% acetic acid in |
| | Phenyl-Hexyl 4.60 mm | water | acetonitrile |
| | diameter × 75 mm | | |
| | length | | |
| 2 | Phenomenex Synergi | 5 mM ammonium | 5 mM ammonium |
| | Polar RP 4.60 mm | acetate in water | acetate in acetonitrile |
| | diameter × 50 mm | | |
| | length | | |
| 3 | Phenomenex Luna | 10 mM ammonium | 10 mM ammonium |
| | Amino (NH2) 4.60 | acetate, 0.25% | acetate, 0.25% |
| | diameter × 50 mm | ammonium hydroxide | ammonium hydroxide |
| | length | in water | in 90% |
| | | | acetonitrile/10% water |

Sample Collection:

Blood: Whole blood, serum or plasma are analyzed.

Whole Blood: Following a finger-stick with a sterile lancet, up to 200 ul of capillary blood are spotted onto a 3 mm disc of S&S Grade 903 filter paper (Schleicher and Schuell), and dried. The disc is then transferred to a well of a deep-well microtiter plate for extraction using methods below.

Serum: Whole blood (50 ul to 5 ml) obtained via finger stick or venous blood draw is allowed to clot at room temperature. Serum is removed from above the clot. Chelating agent and antioxidant are added (EDTA, final concentration 0.4 mM; TEMPO, final concentration 0.8 mM). The sample is extracted immediately, or stored at −80° C. until extraction.

Plasma: Whole blood is collected into an anti-coagulant (heparin or citrate/EDTA). Cells are removed by centrifugation. The plasma layer above the cells is removed to a new tube containing chelating agent and antioxidant are added (EDTA, final concentration 0.4 mM; TEMPO, final concentration 0.8 mM). The sample is extracted immediately, or stored at −80° C. until extraction.

Urine: Urine is collected using a “clean catch” method. The urine is stored as soon as possible at −80° C. Immediately upon thawing, a chelating agent and an anti-oxidant are added to the urine (EDTA, final concentration 0.4 mM; TEMPO, final concentration 0.8 mM). Urine is extracted as described below.

Sample Preparation:

The biological fluids or cells (e.g. a suspension of in vitro cultured cells, blood, urine etc.) arrayed in a 96-well plate are mixed with an equal volume of extraction solvent (e.g. 90/10 Acetonitrile/water, 1% trifluoroacetic acid) and vortexed for 60 seconds. If using soft-tissues (e.g. liver), the tissue is homogenized at 4° C. using a Teflon-on-glass or other appropriate homogenizer in an equal volume of extraction solvent. The resulting solution or homogenate from the above steps is centrifuged at 3,000 g for 15 minutes to remove precipitated proteins and other macromolecules. 100 μl of the supernatant is transferred to a new 96-well plate and dried under Nitrogen The dried sample is then stored at −80° C., until ready for analysis, at which time it is reconstituted with the Internal Standard solution (Stable isotopic and/or deuteriated compounds e.g., Glucose-d7, Valine-d8, glycerol-d8 in 50/50 acetonitrile/water). Alternatively, a biological fluid is used directly, after dilution with the Internal Standard solution.

The platform detects the presence of molecules from a defined list of biochemical compounds (See, e.g., Table 1) and only from this list. Other molecules present in the sample are not detected. This platform is used to create signatures whose components are biochemical compounds that can, in combination, distinguish between classes of samples. Because the identities of the compounds are known, the composition of signatures are subject to biological interpretation.

There are seven components to the platform: 1—8 HPLC pumps used to deliver liquid phases; 2—A 4-injector autosampler for controlling sample injection; 3—up to four different HPLC columns for separation; 4—A switching valve used to control column to MS transfer; 5—An LC/MS interface such as electrospray (ES), atmosphere pressure chemical ionization (APCI) for connection of HPLC and MS; 6—A triple quadrupole mass spectrometer for compound separation and identification; 7—A computer for instrument control and data acquisition.

The columns are indicated in Table 2.

The column switching valve allows staggered injection into the multiple columns, and the effluent from the different columns to be analyzed sequentially in a single run. This way data from 4 columns are captured from single sample on a single mass spectrometer, rather than needing 4 separate runs. Compounds with distinct masses but similar retention times are separated by the mass spectrometer. The targeted compounds (Table 1) are each detected by the MS throughout the run to produce a series of mass chromatograms.

Mass Chromatogram Processing

Biochemical Compound Identification: In order to quantify a single desired biochemical compound, the triple quadrupole mass spectrometer combines two mass filters and a fragmentation step. The first quadrupole acts as a mass filter and only allows ions of a particular mass/charge ratio to proceed further into the second quadrupole. This second chamber acts as the collision cell where the filtered molecules are fragmented with gas molecules and with a source of electrons. This fragmentation causes each parent biochemical molecule to fragment in a predictable manner producing fragment (or daughter) ions of a particular mass. The third quadrupole acts as a second mass filter and only allows the desired daughter ions to pass through to the detector. Thus the combination of the two mass filters allow for quantitation of only molecules with the desired mass/charge ratio that produce daughter ions of the desired mass. In most cases this will detect only a single compound. Distinct biochemical compounds that have identical parent and daughter masses will be ambiguous, and for those situations, it is possible to use the initial step of liquid chromatography to separate the molecules by retention time.

In order to detect and quantify 23 target compounds in a single mass spectrometer run, the parent and daughter ion masses are programmed into the machine. The two mass filters rapidly cycle through these mass combinations, detecting each of the target compounds as the sample comes off the columns.

Biochemical Compound Quantitation: After peak identification, the amount of each compound must be calculated. This is achieved by the step of peak integration. The area under the peak for each of the target compounds is calculated using the AB Analyst software. These values are then scaled by the area of the internal standard peak, producing a relative peak area ratio.

QC: In addition to standard processing, each sample is run through a suite of QC procedures which examine (among other things) retention times, and peak areas for internal standards for indications of problems with the LC/MS process. In addition, individual peaks are flagged for manual examination if parameters (such as for peak shape) exceed normal bounds.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A urinary tract disorder reference profile, comprising a pattern of one or more analytes or metabolites thereof, selected from the group consisting of UTD 3, 6, 8-11, and 18.

2. The urinary tract disorder reference profile of claim 1, further comprising a pattern of one or more analytes or metabolites thereof, selected from the group consisting of UTD 24-95.

3. A urinary tract disorder reference profile, comprising a pattern of one or more analytes or metabolites thereof, selected from the group consisting of UTD 6 and 24-55.

4. A urinary tract disorder reference profile, comprising a pattern of one or more analytes or metabolites thereof, selected from the group consisting of UTD 3, 8-11, 18, 56-95.

5. A method of metabolomically predicting whether a subject is predisposed to developing a urinary tract disorder, comprising obtaining a urinary tract disorder reference profile from said subject and comparing the urinary tract disorder reference profile from said subject with a control urinary tract disorder reference profile, thereby predicting whether the subject is predisposed to having a urinary tract disorder.

6. The method of claim 5, wherein the urinary tract disorder is interstitial cystitis, prostatitis, kidney infection or inflammation, urethritis, prostrate hypertrophy, or urinary tract stones.

7. A method for identifying markers indicative of a urinary tract disorder in a subject comprising determining the levels of one or more analytes or metabolites thereof in a subject sample, wherein said analytes or metabolites are selected from the group consisting of UTD 3, 6, 8-11, and 18 and determining those analytes or metabolites that are present in a different concentration in the subject sample compared to a control sample, wherein the presence of said analytes or metabolite at a different concentration is indicative of a urinary tract disorder in said subject.

8. The method of claim 7, further comprising determining the concentration of one or more analytes or metabolites thereof selected from the group consisting of UTD 24-95.

9. The method of claim 7, wherein said subject sample is urine, prostatic fluid or urinary tract tissue.

10. The method of claim 7, wherein said control sample is derived from a subject known not to be suffering from or pre-disposed to developing a urinary tract disorder.

11. A method of diagnosing a urinary tract disorder (UTD) or a predisposition to developing a urinary tract disorder in a subject, comprising determining a level of a UTD-associated analyte in a subject derived sample, wherein an increase or decrease of said level compared to a normal control level indicates that said subject suffers from or is at risk of developing a urinary tract disorder.

12. The method of claim 11, wherein said UTD-associated analyte is selected from the group consisting of UTD 6 and 24-55, wherein an increase in said level compared to a normal control level indicates that said subject suffers from or is at risk of developing developing a urinary tract disorder.

13. The method of claim 12, wherein said increase is at least 1.1-fold greater than said normal control level.

14. The method of claim 11, wherein said analyte is selected from the group consisting of UTD 3, 8-11, 18 and 56-95, wherein a decrease in said level compared to a normal control level indicates that said subject suffers from or is at risk of developing developing a urinary tract disorder.

15. The method of claim 14, wherein said decrease is at least 10% less than said normal control level.

16. A method of assessing the efficacy of a treatment of a urinary tract disorder in a subject, comprising determining a level of a UTD-associated analyte in a subject sample derived after treatment and comparing said level to a normal control level, thereby monitoring the treatment of the urinary tract disorder in said subject.

17. The method of claim 16, wherein a similarity of said level of said UTD-associated analyte in said subject sample compared to a said normal control level indicates that treatment is efficacious.

18. A method of determining the risk of developing a urinary tract disorder in a subject, comprising detecting an elevated concentration of an analyte or metabolite thereof selected from the group consisting of UTD 6 and 24-55 compared to the concentration of said analyte or metabolite in a control sample, wherein an elevated concentration of said analyte or metabolite indicates said subject is at risk of developing a urinary tract disorder.

19. A method of determining the risk of developing a urinary tract disorder in a subject, comprising detecting a decreased concentration of an analyte or metabolite thereof selected from the group consisting of UTD 3, 8-11, 18 and 56-95 compared to the concentration of said analyte or metabolite in a control sample, wherein a decreased concentration of said analyte or metabolite indicates said subject is at risk of developing a urinary tract disorder.

20. A method of identifying an agent that modulates the onset or progression of a urinary tract disorder in a subject, comprising:
i) contacting said subject with a candidate agent;
ii) determining a test level of an analyte in a sample derived from said subject following said contacting;
iii) comparing said test level with a reference level of said analyte, wherein an increase or decrease of said test level relative to said reference level indicates that said test agent modulates the onset or progression of a urinary tract disorder.

21. The method of claim 20, wherein said reference level is derived from a sample derived from said subject.

22. The method of claim 20, wherein said reference level is derived from a database.

23. The method of claim 22, wherein said database comprises test levels of an analyte in a sample derived from a database subject, wherein said database subject is not said test subject.

24. A kit comprising a detection reagent that identifies one or more analytes selected from the group consisting of UTD 3, 6, 8-11, 18, and 24-95.
